(19) United States
(12) Patent Application Publication
(10) Pub. No.: US 2025/0276160 A1
(43) Pub. Date: Sep. 4, 2025

(54) ADJUSTABLE SHUNTS WITH IMPROVED FLOW CONTROL AND ASSOCIATED SYSTEMS AND METHODS

(71) Applicant: Shifamed Holdings, LLC, Campbell, CA (US)

(72) Inventors: David Batten, San Francisco, CA (US); Tessa Bronez, Campbell, CA (US); Tom Saul, Portland, OR (US); Eric Schultz, Los Altos, CA (US)

(21) Appl. No.: 18/858,641

(22) PCT Filed: May 4, 2023
(86) PCT No.: PCT/US2023/020973

§ 371 Date: Oct. 21, 2024

Related U.S. Application Data
(60) Provisional application No. 63/338,393, filed on May 4, 2022.

(51) Int. Cl. A61M 27/00

(52) U.S. Cl. CPC A61M 27/002

(57) ABSTRACT

The present technology is generally directed to adjustable shunting systems, including adjustable shunting systems with improved flow control. For example, the adjustable shunting systems described herein can include a flow control plate or other feature with a ramping feature that directs a sealing assembly toward and/or at least partially into an inlet or outlet of a drainage channel to improve the seal between the sealing assembly and the inlet or outlet. In some embodiments, the ramping feature is a slot, groove, or other opening in the plate.

### CROSS-REFERENCE TO RELATED APPLICATION(S)

The present application claims priority to U.S. Provisional Patent Application No. 63/338,393, filed May 4, 2022, the disclosure of which is incorporated by reference herein in its entirety.

### TECHNICAL FIELD

The present technology generally relates to implantable medical devices and, in particular, to adjustable shunts for controlling fluid flow between a first body region and a second body region of a patient.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma shunts or “MIGS”) have shown clinical benefit; however, there is a need for improved shunting systems and techniques for addressing elevated intraocular pressure and risks associated with glaucoma, as well as other patient conditions. For example, there is a need for shunting systems capable of adjusting the therapy provided, including the flow rate/fluid resistance between the two fluidly-connected bodies. As another example, there is a need for a shunting system able to be modified after manufacture (e.g., in the clinic) to personalize the system for the patient and/or as part of the clinician's plan for the implant procedure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1A illustrates a shunting system configured in accordance with select embodiments of the present technology.

FIG. 1B illustrates the shunting system of FIG. 1A rotated 180 degrees about its longitudinal axis.

FIG. 1C is an enlarged view of a portion of the shunting system shown in FIGS. 1A and 1B.

FIG. 2A illustrates an actuator for use with the shunting system shown in FIGS. 1A-1C and configured in accordance with select embodiments of the present technology.

FIGS. 2B-2D illustrate embodiments of the sealing assembly and gating element of the actuator shown in FIG. 2A and configured in accordance with select embodiments of the present technology.

FIG. 3 is a schematic illustration of a slot of the shunting system shown in FIGS. 1A-1C and a sealing assembly of the actuator shown in FIG. 2A.

FIGS. 4A1-4C1 illustrate a series of operating stages for adjusting flow through the shunting system of FIGS. 1A-1C in accordance with embodiments of the present technology, and FIGS. 4A2-4C2 illustrate another view of the series of operating stages for adjusting flow through the shunting system of FIGS. 1A-1C shown in FIGS. 4A1-4C1.

FIGS. 5 and 6 illustrate additional configurations of slots for use with shunting systems and configured in accordance with select embodiments of the present technology.

FIG. 7 is an exploded view of another shunting system configured in accordance with select embodiments of the present technology.

### DETAILED DESCRIPTION

The present technology is generally directed to adjustable shunting systems, including adjustable shunting systems with improved flow control. For example, the adjustable shunting systems described herein can include a shunting element with at least one channel extending therethrough that permits fluid to flow through the system. The system can further include an actuator that can be selectively actuated to control the flow of fluid through an inlet or outlet of the channel to titrate the level of therapy provided by the shunt. In some embodiments, the actuator can include a gating element having a sealing assembly moveable between a first (e.g., closed) position in which the sealing assembly provides a first resistance to fluid flow through the inlet or outlet (e.g., by blocking the channel inlet or outlet), and a second (e.g., open) position in which the sealing assembly provides a second resistance less than the first resistance (e.g., by not blocking the channel inlet or outlet).

To further improve the flow control provided by the sealing assembly, the system can include a flow control plate that defines a ramping feature for directing the sealing assembly toward and/or at least partially into the inlet or outlet when the actuator is in the first position. As described throughout this Detailed Description, the ramping feature can be a slot, groove, or opening formed in the flow control plate and that extends at least partially between a first surface and a second opposite surface of the plate. The slot, for example, can be tapered such that it extends between a first relatively narrow end portion and a second relatively wider end portion (e.g., a width of the slot increases from the first relatively narrow end portion to the second relatively wider end portion). The tapered shape of the slot causes the sealing assembly to move in both a “horizontal” direction (e.g., parallel to a plane defined by the plate) and a “vertical” direction (e.g., perpendicular to the plane defined by the plate) as the sealing assembly moves between the first position and the second position. As described in detail throughout this Detailed Description, this multi-plane motion is expected to improve flow control through the system, such as by improving the seal obtained between the sealing assembly and the channel inlet or outlet when the sealing assembly is in the first position.

In addition to or in lieu of the flow control plate with the ramping feature, in some embodiments the system can include a moveable flap proximate the inlet or the outlet. In such embodiments, the actuator can direct the moveable flap into and/or over the inlet or outlet when the actuator (e.g., the sealing assembly) is in the first (e.g., closed) position. When the actuator (e.g., the sealing assembly) is in the second (e.g., open) position, the moveable flap generally does not cover the inlet or outlet. Accordingly, similar to the ramping feature, the moveable flap is expected to improve flow control through the system, such as by improving the seal obtained between the sealing assembly and the channel inlet or outlet when the sealing assembly is in the first position.

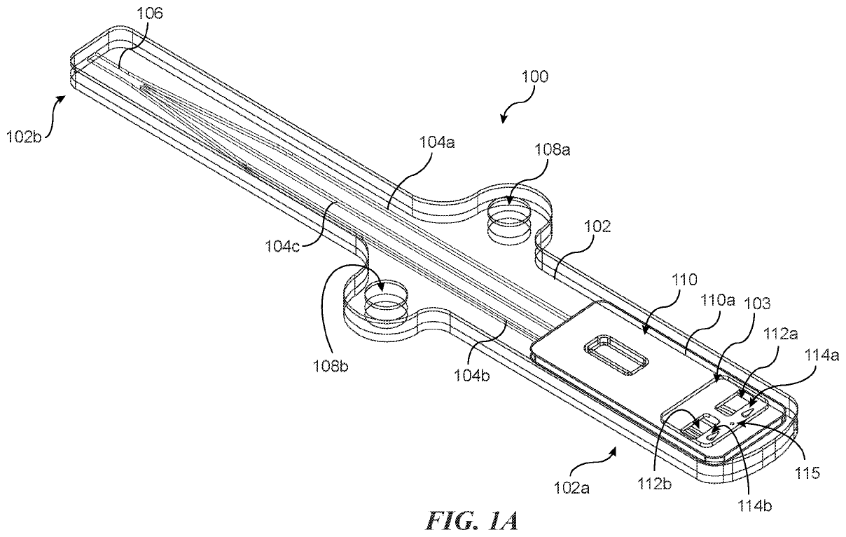

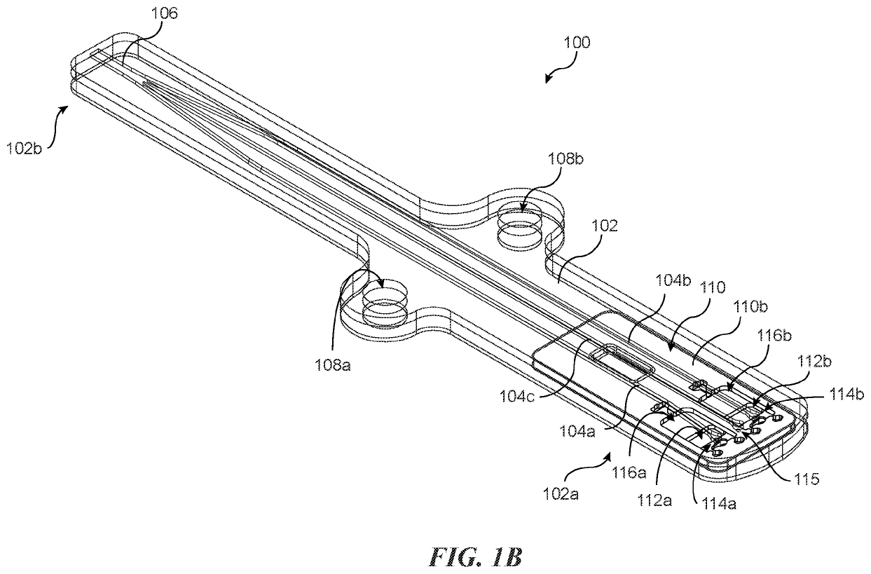

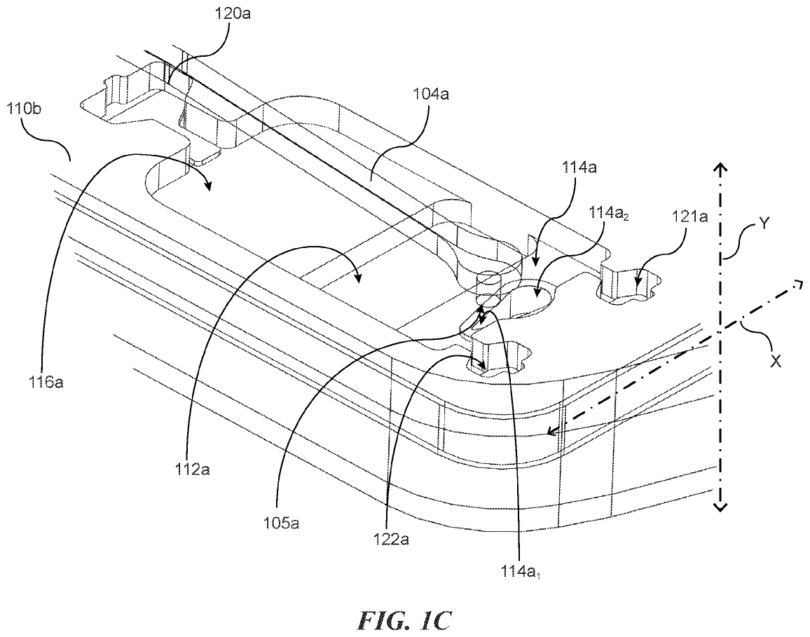

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples and claims but are not described in detail with respect to FIGS. 1A-6.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye, or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A-1C illustrate an adjustable shunting system 100 (“the system 100”) configured in accordance with select embodiments of the present technology. More specifically, FIG. 1A is a perspective view of the system 100, FIG. 1B is a second perspective view of the system 100 with the system 100 rotated 180 degrees about its longitudinal axis relative to the view shown in FIG. 1A, and FIG. 1C is an enlarged view of a portion of the system 100 from the same view shown in FIG. 1B. As described in greater detail below, the system 100 is configured to provide a titratable therapy for shunting fluid from a first body region to a second body region, such as shunting aqueous from an anterior chamber of a patient's eye to a target outflow location.

Referring collectively to FIGS. 1A and 1B, the system 100 includes a shunting element 102 and a plate or cartridge 110. The shunting element 102 (which can also be referred to as a casing, membrane, elongated housing, or the like) extends between a first end portion 102a and a second end portion 102b. As best shown in FIG. 1A, the shunting element 102 can have an opening 103 adjacent the first end portion 102a. A plurality of flow channels 104 (shown as a first channel 104a, a second channel 104b, and a third channel 104c) can extend through the shunting element 102 at least partially between the first end portion 102a and the second end portion 102b. The channels 104 can be fluidly isolated along a portion or substantial portion of the length of the shunting element 102, and can merge into a primary drainage lumen 106 at or proximate the second end portion 102b of the shunting element 102. As described in greater detail below, when the system 100 is implanted within a patient between a first body region and a second body region, fluid can flow from the first body region to the second body region via the channels 104 and the primary drainage lumen 106. The shunting element 102 may optionally include one or more features to facilitate anchoring the system 100 to patient tissue, such as first and second suture holes 108a, 108b. Additional features of shunting elements suitable for use with the present technology are described in International Patent Application No. PCT/US2022/037747, the disclosure of which is incorporated by reference herein in its entirety and for all purposes.

The plate 110 (which can also be referred to as a flow control plate, a flow control cartridge, a flow control feature, etc.) can be positioned within the first end portion 102a of the shunting element 102. As best shown in FIG. 1A (which shows a first or upper surface 110a of the plate 110), the plate 110 can partially align with the opening 103 of the shunting element 102. More specifically, the plate 110 can be positioned within the shunting element 102 such that a first port or opening 112a, a second port or opening 112b, and a third port or opening 115 in the plate 110 align with the opening 103. In operation, fluid can enter the system 100 by flowing through the opening 103 and the first port 112a, the second port 112b, and/or the third port 112c.

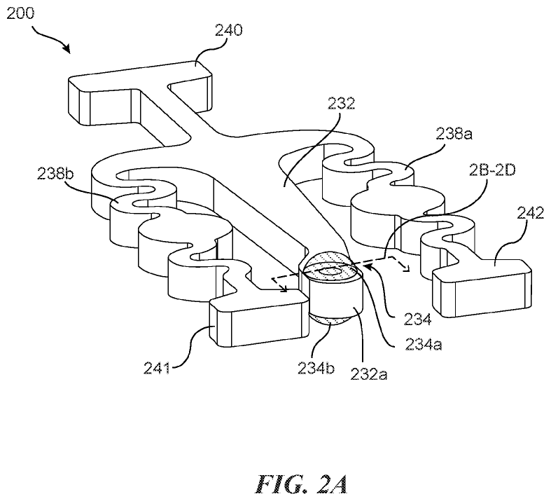

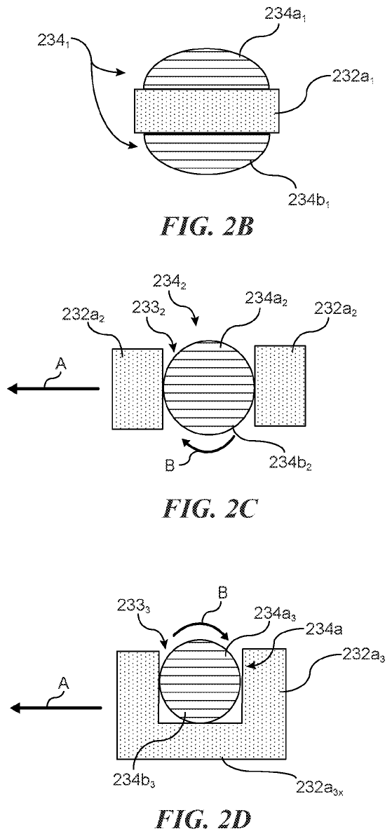

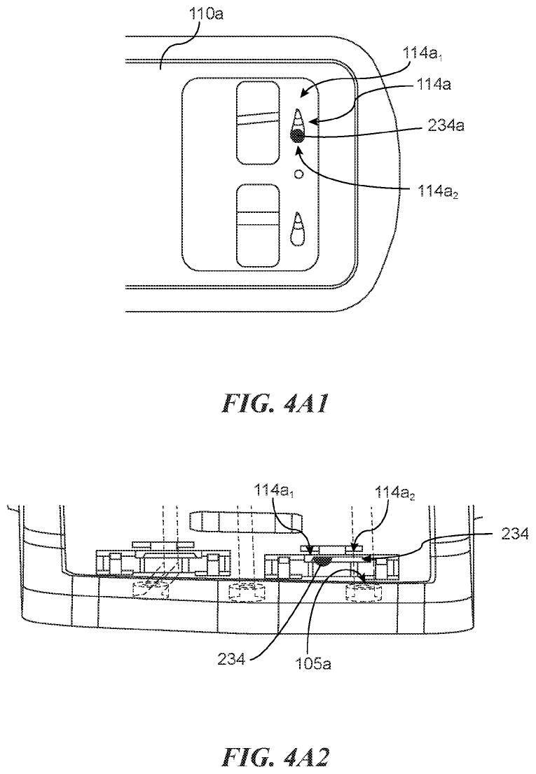

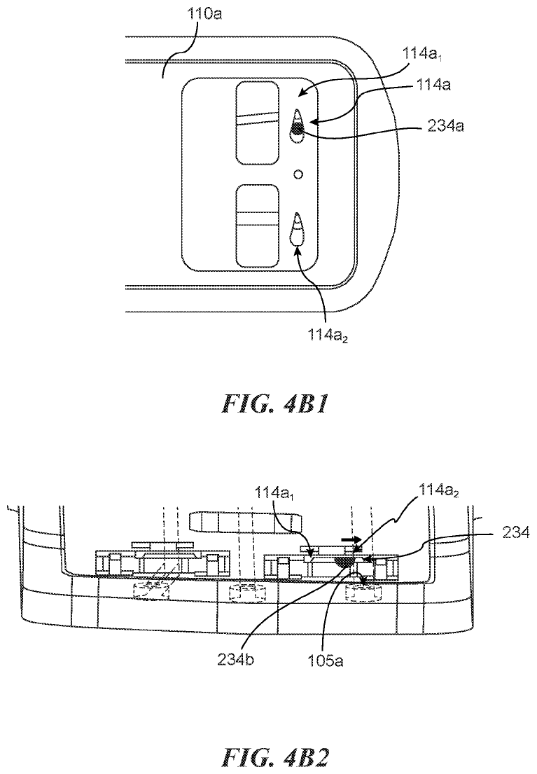

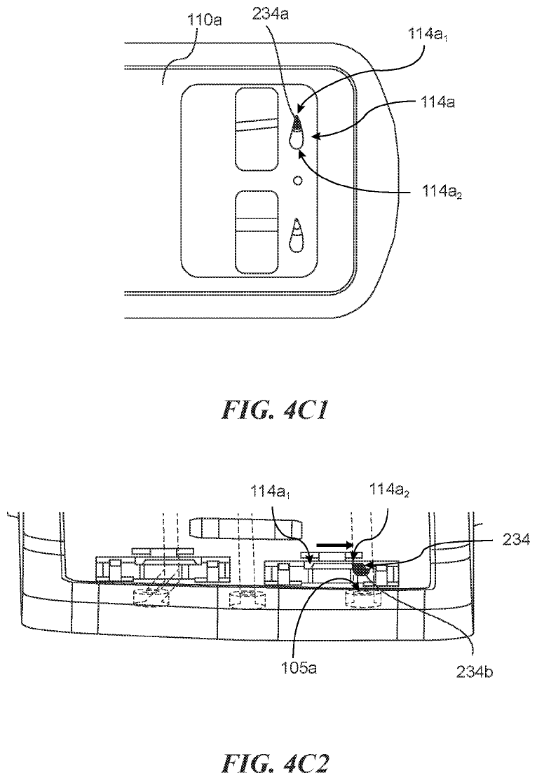

The plate 110 can further include ramping features comprising a first slot 114a and a second slot 114b (collectively referred to herein as “the slots 114”). Each of the slots 114 can be a groove, opening, or other slit that forms an indentation or through-hole in the plate 110. In other embodiments, the slots 114 can be formed by one or more ridges of equal or substantially equal height that extend from a surface of the plate 110. In such embodiments, the slots 114 comprise the “empty space” or chamber formed between the one or more ridges. Regardless of how they are formed, the slots 114 can have a tear-drop shape or other similar shape, such that a “width” of each slot 114 is tapered along the length of each slot 114. As described in greater detail with respect to FIGS. 4A1-4C2, the slots 114 act as a ramp to assist with improving a seal formed between inlets to the channels 104 and a corresponding actuator configured to selectively control flow through the inlets (described with respect to FIG. 2; not shown in FIGS. 1A-1C). In the illustrated embodiment, the slots 114 also align with the opening 103 in the shunting element 102 such that, in embodiments in which the slots 114 extends through the full thickness of the plate 110, fluid may also flow into the system 100 via the slots 114. In other embodiments, however, the slots 114 are not aligned with the opening 103 and do not facilitate fluid flow into the system 100, and instead primarily provide the ramping feature. Regardless, an outer surface of the plate 110 (e.g., the upper surface 110a) generally forms a substantial fluid seal with an inner surface of the shunting element 102 such that fluid does not leak therebetween. As a result, fluid only enters the system 100 via the first port 112a, the second port 112b, and the third port 115 (and the slots 114 in embodiments in which they align with the opening 103 and extend through the full thickness of the plate 110).

As shown in FIG. 1B (which shows a second or lower surface 110b of the plate 110), the plate 110 can further include a first actuator chamber 116a and a second actuator chamber 116b (collectively referred to as “the actuator chambers 116”). The first actuator chamber 116a is fluidly coupled to the first port 112a and the first slot 114a such that fluid flowing through the first port 112a and/or the first slot 114a enters the first actuator chamber 116a. Likewise, the second actuator chamber 116b is fluidly coupled to the second port 112b and the second slot 114b such that fluid flowing through the second port 112b and/or the second slot 114b enters the second actuator chamber 116b. In some embodiments, the actuator chambers 116 are fluidly isolated. In other embodiments, the actuator chambers 116 are fluidly connected. Regardless, the actuator chambers 116 can be configured to receive corresponding actuators (not shown in FIG. 1B; a representative actuator is illustrated in, and described in detail with respect to, FIG. 2) for selectively controlling the flow of fluid through the system 100, as described in greater detail below.

As also shown in FIG. 1B, the channels 104 extend proximate the first end portion 102a of the shunting element 102 such that the plate 110 and the channels 104 at least partially overlap and are in fluid communication. In particular, the first channel 104a overlaps with (and/or is otherwise aligned with) the first actuator chamber 116a, the second channel 104b overlaps with the second actuator chamber 116b, and the third channel 104c interfaces with the third port 115. As described in greater detail with respect to FIG. 1C, the first channel 104a is fluidly coupled with and can be configured to receive fluid from the first actuator chamber 116a via a first aperture or inlet (not shown) and the second channel 104b is fluidly coupled with and can be configured to receive fluid from the second actuator chamber 116b via a second aperture or inlet (not shown). The actuators (not shown) can be configured to selectively and independently interface with the corresponding first and second apertures to control the flow of fluid through the first channel 104a and the second channel 104b.

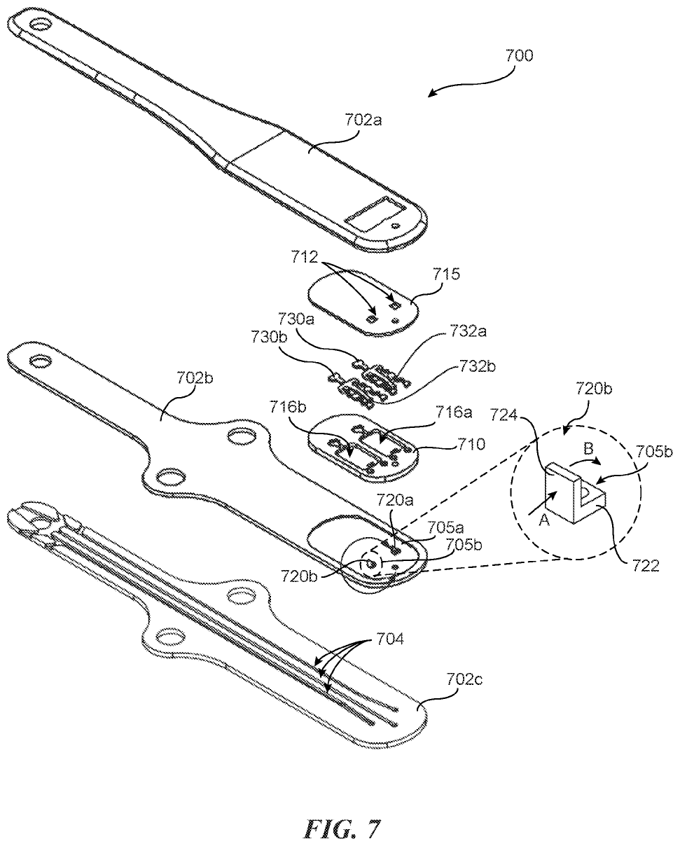

FIG. 1C is an enlarged view of the first actuator chamber 116a and illustrates additional details of the system 100. In particular, FIG. 1C illustrates the first aperture or inlet 105a that fluidly connects the first actuator chamber 116a and the first channel 104a. That is, fluid can flow from the first actuator chamber 116a into the first channel 104a via the first aperture 105a. As illustrated, the first aperture 105a is aligned with (e.g., vertically aligned with) at least a portion of the first slot 114a. In particular, the first aperture 105a is aligned with a first portion 114a1 of the first slot that is relatively narrow (e.g., as opposed to being aligned with a second portion 114a2 of the first slot 114a that is relatively wider than the first portion 114a1). As described in detail with respect to FIGS. 4A1-4C2, aligning the first aperture 105a with the relatively narrow portion 114a1 of the first slot 114a improves the sealing of the first aperture 105a when the actuator (not shown) is transitioned to a “closed” position in which it blocks or substantially blocks fluid from flowing into the first channel 104a via the first aperture 105a. In some embodiments, such as those described in greater detail with reference to FIG. 7, the system 100 can include a hinged or otherwise moveable feature coupled proximate to the first aperture 105a that can also improve the sealing of the first aperture 105a when the actuator is transitioned to the closed position.

As also illustrated in FIG. 1C, the first actuator chamber 116a can also include a first anchoring chamber 120a, a second anchoring chamber 121a, and a third anchoring chamber 122a. As described in greater detail below with reference to FIG. 2, the anchoring chambers 120a-122a are configured to receive and retain corresponding anchoring elements of an actuator such that the actuator is retained within the first actuator chamber 116a.

FIG. 2A illustrates a representative actuator 200 that can be used with the system 100 and is configured in accordance with select embodiments of the present technology. The actuator 200 includes a projection or gating element 232 having a distal end portion 232a configured to at least partially control (e.g., gate) flow through the system 100. To do so, the distal end portion 232a includes a sealing assembly 234 having a first portion 234a positioned on a first (e.g., upper) surface of the distal end portion 232a and a second portion 234b positioned on a second (e.g., lower) surface of the distal end portion 232a. As described in detail below, the first portion 234a is configured to slidably engage the first slot 114a, and can therefore be referred to as a ramping element or feature 234a, while the second portion 234b is configured to releasably engage the first aperture 105a, and can therefore be referred to as a sealing element or feature 234b. As also described in detail below, the sealing assembly 234 is expected to improve the flow-blocking effect of the gating element 232.

The sealing assembly 234 and the distal end portion 232a of the gating element 232 can be designed to interface in a variety of different manners. FIGS. 2B-2D are cross-sectional views of different embodiments of the sealing assembly 234 and distal end portion 232a of the gating element 232 taken along the line 2B-2D shown in FIG. 2A. For ease of reference, FIGS. 2B-2D use the same primary reference numbers to refer to corresponding components shown in FIG. 2A, but add an additional subscript numeral to denote that each component may not be identical in the various embodiments (e.g., in FIG. 2B, the sealing assembly 234 is referred to as the sealing assembly 234₁; in FIG. 2C, the sealing assembly 234 is referred to as the sealing assembly 234₂; etc.).

As shown in FIG. 2B, in some embodiments the first portion 234a₁ and the second portion 234b₁ of the sealing assembly 234₁ are discrete components. In such embodiments, the first portion 234a₁ can be coupled to an upper surface of the distal end portion 232a₁ of the gating element 232 via mechanical tethers, bands, barbs, staples, sutures, glue, chemical bonding, or the like, and the second portion 234b₁ can be separately coupled to a lower surface of the distal end portion 232a₁ via mechanical tethers, bands, barbs, staples, sutures, glue, chemical bonding, or the like. Although shown as having a semi-spherical shape, in other embodiments the first portion 234a₁ and/or the second portion 234a₂ can have other suitable shapes, such as a conical shape.

In other embodiments, the first portion 234a₁ and the second portion 234b₁ are a single unitary component. For example, the first portion 234a₁ and the second portion 234b₁ of the sealing assembly 234₁ can be connected by a medial portion (not shown) that extends through an aperture in the distal end portion 232a₁ of the gating element 232 and that secures the sealing assembly 234₁ to the distal end portion 232a₁. As another example, the first portion 234a₁ and the second portion 234b₁ can be a coating or other material on the surface of the gating element 232 (e.g., completely or at least partially surrounding the distal end portion 232a₁). As yet another example, the sealing assembly 234₁ itself can form the distal end portion 232a₁ of the gating element 232 such that the sealing assembly 234₁ is integral with the gating element 232. In such embodiments, the sealing assembly 234₁ can be generally spherical or conical in shape, with the first portion 234a₁ being a first hemispherical portion of the sphere and the second portion 234b₁ being a second hemispherical portion of the sphere.

In some embodiments, the sealing assembly 234 or a portion thereof is movable relative to (e.g., configured to rotate relative to) the distal end portion 232a of the gating element 232. For example, as shown in FIG. 2C, the sealing assembly 234₂ can have a spherical shape and be positioned within a corresponding cavity or opening 233₂ in the distal end portion 232a₂. A first hemispherical portion 234a2 of the sealing assembly 234₂ forms the “first portion 234a” of the sealing assembly 234₂, and a second hemispherical portion 234b₂ of the sealing assembly 234₂ forms the “second portion 234b” of the sealing assembly 234₂. The sealing assembly 234₂ can be rotatably contained within the distal end portion 232a₂, such that as the distal end portion 232a₂ is translated in a first direction indicated by arrow A, the sealing assembly 234₂ rotates in a second direction indicated by arrow B. Enabling rotation of the sealing assembly 234₂ relative to the distal end portion 232a₂ may improve performance of the actuator 200 because it permits the sealing assembly 234₂ to rotate as it slidably engages the first slot 114a, reducing the frictional interference between the sealing assembly 234₂ and the first slot 114a.

FIG. 2D illustrates another representative embodiment of a sealing assembly 234₃ that is movable relative to (e.g., configured to rotate relative to) a distal end portion 232a₃ of the gating element 232. In the illustrated embodiment, the distal end portion 232a₃ of the gating element 232 forms a cup or recess 233₃ that houses the sealing assembly 234₃. The sealing assembly 234₃ is rotatably moveable relative to the recess 233₃, such that as the distal end portion 232a₃ is translated in a first direction indicated by arrow A, the sealing assembly 234₃ rotates within the recess 233₃ in a second direction indicated by arrow B. In the illustrated embodiment, the first portion 234a₃ is configured to slidably engage the first slot 114a. However, the second portion 234b₃ sits within the recess 233₃ and therefore does not directly engage with the first aperture 105a. Rather, a lower surface 232a₃ₓ of the distal end portion 232a₃ is configured to releasably engage the first aperture 105a.

Returning to FIG. 2A, and regardless of the specific embodiment of the sealing assembly 234 and the distal end portion 232a, the sealing assembly 234 can be at least partially composed of an impermeable and partially compressible or flexible material (e.g., an elastomeric material, a material having a low durometer, a material having a low Young's modulus, etc.). For example, in some embodiments at least the second portion 234b (i.e., the sealing element 234b) is composed of the impermeable and partially compressible material. As described in greater detail below, this enables the second portion 234b to conform to a corresponding channel inlet/aperture to control the flow therethrough. The sealing assembly 234 can also or alternatively be at least partially composed of a generally rigid material (e.g., a non-compressible or non-flexible material having a generally high durometer and/or high Young's modulus). For example, in some embodiments at least the first portion 234a (i.e., the ramping element 234a) is composed of a rigid material that does not substantially deform. Examples of suitable materials having a generally high durometer include, but are not limited to, glass, titanium, Nitinol, stainless steel, high-durometer silicone, high-durometer polymers, or the like. As described in greater detail below, this enables the first portion 234a to slide along the slot 114a without substantially deforming. In some embodiments, both the first portion 234a and the second portion 234b are composed of the rigid material that does not substantially deform.

The sealing assembly 234 can have a dimension (e.g., a height, width, diameter, etc.) that is between about 25 microns and 200 microns, or between about 50 microns and 175 microns, or between about 70 microns and about 150 microns. In some embodiments, a largest dimension (e.g., whichever “edge-to-edge” dimension of height, width, or diameter is greatest) is less than about 150 microns, less than about 125 microns, less than about 100 microns, less than about 90 microns, less than about 80 microns, or less than about 70 microns. The foregoing ranges are provided by way of example only—in some embodiments, the sealing assembly 234 can have dimensions outside of the foregoing ranges.

The actuator 200 further includes a first actuation element 238a and a second actuation element 238b. The first actuation element 238a can be configured to rotate, pivot, slide, or otherwise move the gating element 232, and thus the sealing assembly 234, in a first direction. The second actuation element 238b can be configured to selectively rotate, pivot, slide, or otherwise move the gating element 232, and thus the sealing assembly 234, in a second direction generally opposite the first direction. For example, the first actuation element 238a and the second actuation element 238b can be composed at least partially of a shape memory material or alloy (e.g., nitinol). Accordingly, the first actuation element 238a and the second actuation element 238b can be transitionable at least between a first material phase or state (e.g., a martensitic state, a R-phase, a composite state between martensitic and R-phase, etc.) and a second material phase or state (e.g., an austenitic state, an R-phase state, a composite state between austenitic and R-phase, etc.). In the first material state, the first actuation element 238a and the second actuation element 238b may have reduced (e.g., relatively less stiff) mechanical properties that cause the actuation elements to be more easily deformable (e.g., compressible, expandable, etc.) relative to when the actuation elements are in the first material state. In the second material state, the first actuation element 238a and the second actuation element 238b may have increased (e.g., relatively more stiff) mechanical properties relative to the first material state, causing an increased preference toward a specific preferred geometry (e.g., original geometry, manufactured or fabricated geometry, heat set geometry, etc.). The first actuation element 238a and the second actuation element 238b can be selectively and independently transitioned between the first material state and the second material state by applying energy (e.g., laser energy, electrical energy, etc.) to the first actuation element 238a or the second actuation element 238b to heat it above a transition temperature (e.g., above an austenite finish (Af) temperature, which is generally greater than body temperature). If the first actuation element 238a (or the second actuation element 238b) is deformed relative to its preferred geometry when heated above the transition temperature, the first actuation element 238a (or the second actuation element 238b) will move to and/or toward its preferred geometry. In some embodiments, the first actuation element 238a and the second actuation element 238b are operably coupled such that, when the actuated actuation element (e.g., the first actuation element 238a) transitions toward its preferred geometry, the non-actuated actuation element (e.g., the second actuation element 238b) is further deformed relative to its preferred geometry. Additional details regarding, and examples of, bi-directional shape memory actuators are described in U.S. Patent Application Publication Nos. 2020/0229982 and 2021/0251806, the disclosures of which are incorporated by reference herein in their entireties and for all purposes.

The actuator 200 further includes a first anchoring element 240, a second anchoring element 241, and a third anchoring element 242. To couple the actuator 200 to the system 100, the first anchoring element 240 of the actuator 200 can be secured within the first anchoring chamber 120a (FIG. 1C) of the first actuator chamber 116a, the second anchoring element 241 can be secured within the second anchoring chamber 121a (FIG. 1C) of the first actuator chamber 116a, and the third anchoring element 242 can be secured within the third anchoring chamber 122a (FIG. 1C) of the first actuator chamber 116a. In some embodiments, the first actuation element 238a and the second actuation element 238b are deformed relative to their preferred geometries (e.g., “loaded”) when the actuator 200 is positioned within the corresponding actuator chamber of the system 100 (e.g., the first actuator chamber 116a; FIG. 1C). For example, the anchoring chambers 120a-122a can be configured/dimensioned such that the act of placing the corresponding anchoring elements 240-242 within the anchoring chambers 120a-122a deforms the first actuation element 238a and the second actuation element 238b relative to their original manufactured geometries. In some embodiments positioning the anchoring elements 240-242 within corresponding anchoring chambers 120a-122a can increase a length of the actuation elements 138 (e.g., tension) relative to their preferred geometries. In other embodiments, positioning the anchoring elements 240-242 within corresponding anchoring chambers 120a-122a can decrease a length of the actuation elements 138 (e.g., compress) relative to their preferred geometries. Additional details regarding loading and deforming shape memory actuators are described in U.S. Patent Application Publication No. US 2021/0251806, previously incorporated by reference herein, and International Patent Application No. PCT/US21/49140, the disclosure of which is incorporated by reference in its entirety and for all purposes.

The distal end portion 232a of the gating element 232 is configured to moveably interface with various features of the system 100 to at least partially control the flow of fluid through one or more flow pathways extending through the system 100. For example, referring collectively to FIGS. 1C and 2A, when the actuator 200 is positioned within the first actuator chamber 116a, the distal end portion 232a of the gating element 232 is positioned generally between the first aperture 105a and the first slot 114a. As a result, the first portion 234a (i.e., the ramping element 234a) of the sealing assembly 234 is configured to slidably engage the first slot 114a, and the second portion 234b (i.e., the sealing element 234b) of the sealing assembly 234 is configured to releasably engage the first aperture 105a.

The actuation elements 238 are configured to translate the sealing assembly 234 in a generally “horizontal” direction along a length of the slots 114 (e.g., within a plane parallel to a plane defined by the plate, labeled in FIG. 1C as the axis X). Depending on the configuration of the actuator 200, the actuation elements 238 may translate the sealing assembly 234 along an arc as opposed to along a linear path (e.g., in embodiments in which the actuation elements 238 cause the gating element 232 to rotate or pivot). Such translation still occurs within the plane parallel to the plane defined by plate, just along an arcuate path instead of a linear path. In such embodiments, the slot 114 can be at least partially curved (e.g., a curved tear-drop shape) such that the slot 114 traces the sealing assembly's translation arc.

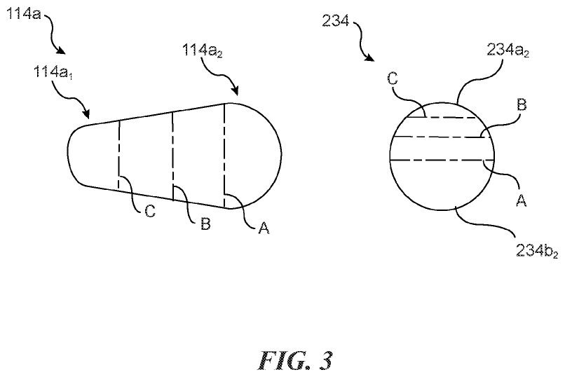

Additionally, as illustrated and described below with reference to FIGS. 4A1-4C2, as the sealing assembly 234 is horizontally translated via actuation of the actuation elements 238, the slots 114 act as “two-dimensional ramps” that change a “vertical” position of the sealing assembly 234 (e.g., within a plane normal to the plane defined by the plate, labeled in FIG. 1C as the axis Y). In particular, the variable/tapered width of the slots 114 affect how much of the sealing assembly 234 can “fit” within the slot 114, and thus affect a vertical position of the sealing assembly 234. For example, FIG. 3 schematically illustrates a top view of the first slot 114a and a side view of the sealing assembly 234. As seen, the second portion 114a₂ of the slot 114 has a first width A that is equal or approximately equal to a first dimension A (i.e., the diameter) of the sealing assembly 234, a middle portion of the first slot 114a has a second width B that is less than the first width A and is equal or approximately equal to a second dimension B of the sealing assembly 234, and the first portion 114a₁ of the first slot 114a has a third width C that is less than the first width A and the second width B, and that is equal or approximately equal to a third dimension C of the sealing assembly 234. As a result, when the sealing assembly 234 is aligned with the second, relatively wider, portion 114a₂, nearly half of the sealing assembly 234 can fit within the slot 114a. However, when the sealing assembly 234 is aligned with the first, relatively narrower, portion 114a₁ of the first slot 114a, only a small portion of the sealing assembly 234 can fit within the first slot 114a. To prevent the sealing assembly 234 from fully “falling into” or otherwise fitting within the first slot 114a, the largest dimension of the sealing assembly 234 (e.g., the largest diameter) can be at least slightly larger than the largest dimension (e.g., width) of the first slot 114a.

Accordingly, referring collectively to FIGS. 1C-3, as the sealing assembly 234 moves from the second, relatively wider, portion 114a₂ of the first slot 114a toward the first, relatively narrow, portion 114a₁ of the first slot 114a (e.g., via actuation of the first actuation element 238a), less of the first portion 234a of the sealing assembly 234 can fit within the first slot 114a. This causes the sealing assembly 234 to be pushed “out of” the first slot 114a in a first vertical direction perpendicular to the plane of the plate 110 in which the first slot 114a is formed (e.g., in a direction vertically away from the slot 114a, such as along the axis Y labeled in FIG. 1C). And because the first, relatively narrow, portion 114a₁ of the first slot 114a is vertically aligned with the first aperture 105a, this first vertical movement of the sealing assembly 234 away from slot 114a causes the second portion 234b of the gating element 232 to move toward and/or at least partially into the first aperture 105a, thereby improving the fluidic seal formed between the gating element 232 and the first aperture 105a. The slot 114a can therefore direct the second portion 234b of the gating element 232 to abut, conform to, and/or be partially inserted into the first aperture 105a.

In reverse, as the sealing assembly 234 moves from the first, relatively narrower, portion 114a₁ of the slot 114a toward the second, relatively wider, portion 114a₂ of the first slot 114a (e.g., via actuation of the second actuation element 238b), more of the first portion 234a of the sealing assembly 234 can fit within the first slot 114a. This causes the sealing assembly 234 to move in a second direction perpendicular to the plane of the plate (e.g., in a direction vertically toward/into the first slot 114a, such as also along the axis Y labeled in FIG. 1C) that is opposite the first direction described above. This increases the vertical distance between the second portion 234b of the sealing assembly 234 and the aperture 105a, causing the sealing assembly 234 to disengage from the first aperture 105a and permit fluid to flow therethrough.

Accordingly, the sealing assembly 234 moves in two directions during actuation: (1) horizontally/laterally relative to the plate 110 (e.g., along the axis X shown in FIG. 1C), caused by actuation of a corresponding actuation element 138, and (2) vertically/perpendicular to the plane of the plate 110 (e.g., along the axis Y shown in FIG. 1C), caused by the sealing assembly 234 falling into or climbing out of the first slot 114a. Accordingly, the net motion of the sealing assembly 234 is angled relative to the plate 110 by virtue of the first slot 114a. In this way, the first slot 114a acts as a “two-dimensional ramp,” despite the first slot 114a not actually having a surface that is sloped relative to the plate 110.

FIGS. 4A1-4C1 are a series of top views illustrating movement of the first portion 234a (i.e., the ramping element 234a) of the sealing assembly 234 relative to the first slot 114a, and FIGS. 4A2-4C2 are a series of slightly offset and elevated end views corresponding to the tops views shown in FIGS. 4A1-4C1, respectively. In particular, FIGS. 4A1 and 4A2 illustrate a first position in which the first portion 234a of the sealing assembly 234 is aligned with the second, relatively wider, portion 114a₂ of the first slot 114a. FIGS. 4B1 and 4B2 illustrate a second (e.g., intermediate) position as the sealing assembly 234 moves from the second, relatively wider portion 114a₂ toward the first, relatively narrower, portion 114a₁ of the first slot 114a, such as during actuation of the first actuation element 238a. FIGS. 4C1 and 4C2 illustrate a third position in which the first portion 234a of the sealing assembly 234 is aligned with the first, relatively narrower, portion 114a₁ of the first slot 114a.

In the first position (FIGS. 4A1 and 4A2), the first portion 234a of the sealing assembly 234 can sit at least partially within the first slot 114a, which causes the sealing assembly 234 to be positioned vertically away from the first aperture 105a (best shown in FIG. 4A2). However, as the sealing assembly 234 is moved toward the third position shown in FIGS. 4C1 and 4C2 (i.e., from the second portion 114a₂ of the slot 114 toward the first portion 114a₂ of the slot 114), less of the first portion 234a of the sealing assembly 234 “fits” within the first slot 114a. This causes the sealing assembly 234 to be pushed vertically away from the first slot 114a as it moves from the first position toward the third position. As previously described, the first aperture 105a is vertically aligned with the first portion 114a₁ of the first slot 114a. Thus, the vertical movement of the sealing assembly 234 caused by the first slot 114a as the sealing assembly 234 moves from the first position toward the third position directs the sealing assembly 234 (and more specifically, the second portion 234b of the sealing assembly) into the first aperture 105a, thereby improving the seal between the sealing assembly 234 and the first aperture 105a (for purposes of illustration, the second portion 234b is shown as slightly spaced apart from the first aperture 105a; one skilled in the art will appreciate from the disclosure herein that the second portion 234b can abut and even at least partially sit within the first aperture 105a when in the third position illustrated in FIGS. 4C1 and 4C2). The operation can be reversed to “open” the first aperture 105a (e.g., the sealing assembly 234 can be translated from the third position toward the first position) by actuating the other actuation element.

Flow through the second channel 104b can be controlled in the same or generally similar manner as flow through the first channel 104a. For example, the system 100 can include a second actuator 200 positioned in the second actuator chamber 116b that selectively interferes with a second aperture or inlet to the second channel 104b. The second slot 114b can perform the same or similar function as described above with respect to the first slot 114a, but relative to the second aperture or inlet. That is, the second slot 114b can act as a two-dimensional ramp to improve flow control through the second channel 104b. In contrast to the first channel 104a and the second channel 104b, the third channel 104c is designed to be “always open” such that it permits at least some degree of fluid flow through the system 100 even when both the first channel 104a and the second channel 104b are blocked/closed. Of course, the present technology is not limited to particular combinations of “always open” and adjustable channels, and can include more or fewer of each channel type.

Moreover, although FIGS. 1A-4C2 show and describe the slot 114a as being “above” the actuator 200 and the channel inlet (i.e., the first aperture 105a) as being “below” the actuator 200, the relative positioning of these features can be reversed, such that the slot 114a is “below” the actuator 200 and the channel inlet (i.e., the first aperture 105a) is “above” the actuator. In such embodiments, the slot 114a can be downstream of the first aperture 105a (e.g., as opposed to being upstream of the first aperture 105a, as described with reference to FIGS. 1A-4C2).

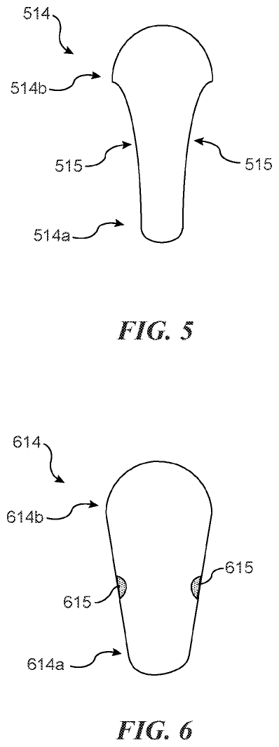

The slots described herein can have other suitable shapes and configurations while still acting as a “two-dimensional” ramp. For example, FIG. 5 illustrates a slot 514 configured in accordance with select embodiments of the present technology. Similar to the slot 114, the slot 514 extends between a first, relatively narrow, end portion 514a and a second, relatively wider, end portion 514b. However, unlike the slot 114 which is formed by two side walls that are relatively straight, the slot 514 is formed in part by two partially curved side walls 515. As a result, the width of the slot 514 does not increase linearly between the first end portion 514a and the second end portion 514b. As one skilled in the art will appreciate from the disclosure herein, changing the tapering of the slot 514 will change the vertical vs. horizontal movement of the sealing assembly (not shown) as it moves between the first end portion 514a and the second end portion 514b. For example, the shape of the slot 514 can be manipulated to control the relative degree of horizontal versus vertical translation. In some embodiments, the sealing assembly (not shown) can have an irregular shape or curvature that is designed to control the relative degree of horizontal versus vertical translation, in addition to or in lieu of the slot 514 being manipulated to control the relative degree of horizontal versus vertical translation.

FIG. 6 illustrates another slot 614 configured in accordance with select embodiments of the present technology. The slot 614 is generally similar to the slots 114, but further includes one or more frictional or resistive elements 615. The frictional or resistive elements 615 can be ridges, bumps, projections, or the like. Alternatively, the frictional elements 615 can be a recess or other widening in the width of the slot 614. In operation, the frictional elements 615 at least partially restrict movement of the sealing assembly (not shown) as it moves between the first end portion 614a and the second end portion 614b. However, the frictional elements 615 do not interfere with the sealing assembly to the extent that it is permanently prevented from moving between the first end portion 614a and the second portion 614b. Rather, the frictional elements 615 reduce or prevent unwanted movement of the sealing assembly, but permit the sealing assembly to move in response to the corresponding actuator (not shown) coupled to the sealing assembly being actuated. In other words, moving the sealing assembly 234 past the friction elements 615 requires an energy input (e.g., via actuation of the actuator) and helps prevent and/or at least reduce unwanted or passive movement of the sealing assembly 234. Examples of using frictional elements to improve the operation of adjustable shunting systems are described in International Patent Application No. PCT/US21/49140, the disclosure of which was previously incorporated by reference herein.

Without being bound by theory, the slots described herein (e.g., the slots 114, 514, and 614) are expected to improve performance of adjustable shunting systems, such as the system 100. In particular, the slots are expected to reduce or minimize leakage of fluid through the corresponding channel inlet (e.g., the first aperture 105a) when a user sets the actuator to the “closed” position. That is, the slots improve the degree of control over fluid flow through adjustable shunting systems by providing a better “seal” when the actuator is moved to the “closed” position. In some embodiments, however, a small, fixed leak through the corresponding inlet may still exist even when the actuator has been moved to the “closed” position.

An additional expected advantage of the present technology is that, in certain embodiments and depending on the manufacturing methods utilized to make the system, the slots are expected to be relatively easier to manufacture than “three-dimensional” ramps that have a sloped surface extending from the plate. For example, removing material from the plate to form the slot can be simpler than adding material to form a sloped three-dimensional ramp. Of course, the slots and other ramping features described herein provide other advantages beyond those expressly described herein, and the present technology is therefore not limited by the foregoing expected advantages. Similarly, the slots or other similar ramping features may be incorporated into adjustable shunting systems other than those expressly described herein.

The systems described herein can have other features expected to improve the sealing ability of the actuator, in combination with or in lieu of the slots 114. For example, FIG. 7 is an exploded view of another adjustable shunting system (“the system 700”) configured in accordance with select embodiments of the present technology. The system 700 can include certain features that are generally similar to some of the features of the system 100 described above. Accordingly, the description below generally focuses on features of the system 700 that differ from the system 100. However, one skilled in the art will appreciate from the disclosure herein that various aspects of the system 100 can be incorporated into the system 700, and vice versa.

The system 700 includes a shunting element 702 comprising a first layer 702a, a second layer 702b, and a third layer 702c. When assembled, a lower surface of the first layer 702a is coupled (e.g., adhered, bonded, etc.) to an upper surface of the second layer 702b, and a lower surface of the second layer 702b is coupled to the upper surface of the third layer 702c. The third layer 702c defines a plurality of channels 704 for permitting fluid to flow through the system 700, similar to the channels 104 of the system 100. Additional details regarding multi-layered shunting elements are described in International Patent Application Publication No. WO 2023/004067, the disclosure of which is incorporated by reference herein in its entirety.

The system 700 further includes a first plate or cartridge 710. The first plate 710 can be generally similar to the plate 110 described with reference to FIGS. 1A-1C. For example, the first plate 710 can include a first chamber 716a for receiving a first actuator 730a and a second chamber 716b for receiving a second actuator 730b. Relative to the plate 110 of FIGS. 1A-1C, the first plate 710 does not necessarily include the slot 114. Instead, as described below, the system 700 has a different mechanism for improving the sealing-capability of the first actuator 730a and the second actuator 730b. The system 700 can optionally include a second plate 715 that forms a “top” to the first plate 710 and holds the first actuator 730a and the second actuator 730b in the corresponding chambers 716a/b of the first plate 710. The second plate 715 includes a plurality of apertures 712 that permit fluid to flow through the second plate 715 (e.g., toward the channels 704).

The first actuator 730a and the second actuator 730b (collectively referred to herein as “the actuators 730”) can be generally similar to the actuator 200 described with reference to FIGS. 2A-2D. For example, the first actuator 730a can include a first gating element 732a and the second actuator 730b can include a second gating element 732b (collectively, “the gating elements 732”). However, in some embodiments the actuators 730 do not have a “sealing assembly,” such as a spherical element, at the distal ends of their respective gating elements 732, as described above with reference to FIGS. 2A-2D. In other embodiments, the actuators 730 include sealing assemblies similar to or the same as those described with reference to FIGS. 2A-2D. Regardless of whether the actuators 730 have sealing assemblies or not, the actuators 730 can be configured to selectively control the flow of fluid through the system 700, as described in further detail below. When the system 700 is assembled, the actuators 730 sit within the corresponding chambers 716 of the first plate 710 and “below” the second plate 715. The first plate 710, the second plate 715, and the actuators 730 are positioned between the first layer 702a and the second layer 702b.

The second layer 702b can include a first aperture 705a and a second aperture 705b (collectively, “the apertures 705”) for permitting fluid to flow through the second layer 702b and into the corresponding channels 704. Each aperture 705 can comprise a corresponding sealing assembly valve 720, shown as a first sealing assembly valve 720a and a second sealing assembly valve 720b (collectively, “the valves 720”). As best seen in the enlarged cut-away view of the second sealing assembly valve 720b, the second valve 720b includes a base portion 722 and a flap portion 724 (which can also be referred to simply as a flap, appendage, moveable feature, etc.). The second aperture 705b is formed in the base portion 722. Although described as a base portion 722, one skilled in the art will appreciate that the base portion 722 can simply be a portion of the second layer 702b that defines the aperture 705b. Accordingly, the flap portion 724 can extend from a surface of the second layer 702b.

The flap portion 724 can hinge, bend, flex, or otherwise move relative to the base portion 722. For example, in response to a force applied against the flap portion 724 in the direction A, the flap portion 724 can hinge or otherwise fold in the direction B from a first position (illustrated) in which it does not block the aperture 705 toward a second position (not shown) in which the flap portion 724 is aligned with the base portion 722 and covers or blocks the aperture 705b. To facilitate the relative motion between the flap portion 724 and the base portion 722, the valve 720b can be composed of an at least partially flexible material such as silicone. In other embodiments, the valve 720b can be made of a generally rigid material with a hinge connecting the flap portion 724 to the base portion 722. Regardless, the valve 720b can be biased toward the first position (illustrated) in which it does not block the aperture 705b (e.g., the valve 720b generally does not block the aperture 705b unless or until an external force moves the flap portion 725 into the second position). In the first position, an angle between the flap portion 724 and the base portion 722 can be between about 60 degrees and about 120 degrees, or between about 70 degrees and about 110 degrees, or between about 80 degrees and about 100 degrees, or about 90 degrees (e.g., the flap portion 724 is generally perpendicular to the base portion 722). In the second position, an angle between the flap portion 724 and the base portion 722 can be less than about 20 degrees, less than about 10 degrees, less than about 5 degrees, or about 0 degrees (e.g., the flap portion 724 is generally parallel to the base portion 722).

In operation, the valves 720 assist with “sealing” or otherwise preventing fluid from flowing through the corresponding apertures 705 when desired. For example, similar to described above with reference to FIG. 2A, the gating elements 732 can be selectively moved by actuating the actuators 730. To close the valve 720b and stop fluid flow through the aperture 705b, the corresponding actuator 730b can be actuated to move the gating element 732b in the direction A until the gating element 732b contacts the flap portion 724 and causes it to bend in the direction B, such that the flap portion 724 blocks the aperture 705b. To open the valve 720b and permit fluid flow through the aperture 705b, the corresponding actuator 730b can be actuated to move the gating element 732b in a direction opposite the direction A until the gating element 732b no longer contacts the flap portion 724, such that the flap portion 724 resiliently springs back toward the illustrated configuration in which it does not cover the aperture 705b. In this way, the actuators 730 and the valves 720 operate to selectively control the flow of fluid through the apertures 705. And because the apertures 705 act as a fluid inlet to the channels 704, the actuators 730 and the valves 720 can be used to selectively control the flow of fluid through the channels 704.

Although the foregoing generally describes the second valve 720b and the second actuator 730b, the description applies equally to the first valve 720a and the first actuator 730b. Similarly, although the system 700 is shown without a slot, the system 700 can include a slot in addition to the valves 720. In such embodiments, the slot(s) can further assist with moving the valves into the second (e.g., closed) position by inducing vertical movement of the respective gating element, as described in detail previously. Accordingly, one skilled in the art will appreciate that the embodiments shown and described herein can be combined to form other embodiments with features for improving control of fluid flow through shunts.

The systems described herein can be designed for shunting fluid between a variety of body regions. For example, in some embodiments the systems described herein are designed to be implanted in a patient's eye to shunt aqueous between the anterior chamber and a target outflow location (e.g., a subconjunctival bleb space), such as to treat glaucoma. Accordingly, in some embodiments the systems described herein can have dimensions compatible with being implanted in the patient's eye. For example, the systems described herein (e.g., the system 100) may have a length of between about 4 mm and about 20 mm, such as between about 4 mm and 15 mm, or between about 4 mm and 12 mm, or between about 6 mm and 10 mm, or about 8 mm. In some embodiments, the plates described herein (e.g., the plate 110) can have a width or thickness less than about 500 microns, less than about 400 microns, less than about 300 microns, and/or less than about 200 microns. In some embodiments, the diameter of the fluidic channels and corresponding apertures (e.g., the channels 104 and the inlets 105) may be less than about 100 microns, less than about 75 microns, and/or less than about 50 microns, such as about 35 microns. Of course, the foregoing dimensions are provided by way of example only, and other dimensions outside the ranges provided above are possible and included within the scope of the present technology. Indeed, the dimensions of the systems described herein may be designed depending on the type of shunting system (e.g., glaucoma shunt vs. hydrocephalus shunt) and intended recipient (e.g., child vs. adult).

### EXAMPLES

Several aspects of the present technology are set forth in the following examples:
1. An adjustable shunting system for shunting fluid from a first body region to a second body region within a patient, the system comprising:
   a shunting element configured to extend at least partially between the first body region and the second body region of the patient;
   a channel extending at least partially through a length of the shunting element between the first body region and the second body region, the channel including an aperture;
   a flow control plate having a slot therein, the slot extending between a first end portion having a first width and a second end portion having a second width greater than the first width such that a width of the slot increases from the first end portion to the second end portion, wherein the first end portion is at least partially vertically aligned with the aperture; and
   an actuator at least partially extending between the slot and the aperture, wherein the actuator includes a first portion configured to slidably engage the slot and a second portion configured to releasably engage the aperture.
2. The system of example 1 wherein the actuator is moveable between (a) a first position in which the first portion of the actuator is adjacent the first end portion of the slot and (b) a second position in which the second portion of the actuator is adjacent the second end portion of the slot, and wherein:
   in the first position, the second portion of the actuator at least partially blocks fluid flow through the aperture, and
   in the second position, the second portion of the actuator does not block fluid flow through the aperture.
3. The system of example 2 wherein the slot is configured to direct the second portion of the actuator toward the aperture as the actuator is moved from the second position toward the first position.
4. The system of example 2 or example 3 wherein the slot is configured to direct the second portion of the actuator away from the aperture as the actuator is moved from the first position toward the first position.
5. The system of any of examples 2-4 wherein the first portion and the second portion of the actuator move (i) in a horizontal direction parallel to a plane of the flow control plate and (ii) a vertical direction normal to the plane of the flow control plate.
6. The system of any of examples 2-4 wherein the actuator is selectively actuatable to move between the first position and the second position, and wherein the actuator is configured such that, following actuation, the actuator retains its position.
7 The system of any of examples 1-6 wherein the slot is an indentation in the flow control plate.
8. The system of any of examples 1-6 wherein the slot is an opening extending between opposing surfaces of the flow control plate.
9. The system of any of examples 1-6 wherein the slot is formed between one or more ridges extending from a surface of the flow control plate.
10. The system of any of examples 1-9 wherein the slot is tear-drop shaped.
11. The system of any of examples 1-10 wherein the slot includes a first side wall and a second side wall, and wherein the first side wall and the second side wall are straight.
12. The system of any of examples 1-10 wherein the slot includes a first side wall and a second side wall, and wherein the first side wall and the second side wall are curved.
13. The system of any of examples 1-12 wherein the slot includes one or more frictional elements configured to at least partially restrict movement of the first portion of the actuator within the slot.
14. The system of any of examples 1-13 wherein the actuator includes a sealing assembly, and wherein the sealing assembly includes the first portion and the second portion.
15. The system of example 14 wherein the first portion of the sealing assembly and the second portion of the sealing assembly form a single, integral component.
16. The system of example 14 wherein the sealing assembly has a spherical shape, and wherein the first portion is a first hemispherical portion of the spherical shape and the second portion is a second hemispherical portion of the spherical shape.
17. The system of example 16 wherein the actuator further comprises a gating element having a cavity extending therethrough, and wherein the sealing assembly is positioned within the cavity and configured to rotate relative to the cavity.
18. The system of example 14 wherein the first portion of the sealing assembly and the second portion of the sealing assembly are distinct, separate components.
19. The system of example 18 wherein the first portion is composed of a first material, and wherein the second portion is composed of a second material different than the first material.
20. The system of example 19 wherein the first material has a higher durometer than the second material.
21. The system of any of examples 1-13 wherein the actuator includes a sealing assembly and a gating element, and wherein the sealing assembly includes the first portion and the gating element includes the second portion.
22. The system of example 21 wherein the actuator includes a recess and the sealing assembly comprises a spherical shape, and wherein the sealing assembly sits at least partially within the recess and is configured to rotate relative to the recess.
23. The system of any of examples 1-22 wherein the actuator includes a sealing assembly and a gating element, and wherein the sealing assembly is configured to rotate relative to the gating element.
24. The system of any of examples 1-23 wherein the system is configured to shunt aqueous from an anterior chamber of the patient's eye to a target outflow location within the patient.
25. An adjustable shunting system for shunting fluid from a first body region to a second body region within a patient, the system comprising:
   a shunting element configured to extend at least partially between the first body region and the second body region of the patient, the shunting element having a channel extending at least partially therethrough;
   an aperture configured to permit fluid to flow into or out of the channel;
   a moveable flap proximate the aperture; and
   an actuator configured to selectively move the moveable flap from a first position in which it does not block the aperture to a second position in which it at least partially blocks the aperture.
26. The system of example 25 wherein the moveable flap extends from a base portion, and wherein the base portion defines the aperture.
27. The system of example 26 wherein, when the moveable flap is in the first position, an angle between the moveable flap and the base portion is between about 60 degrees and 120 degrees.
28. The system of example 25 or example 26 wherein, when the moveable flap is in the second position, an angle between the moveable flap and the base portion is less than about 10 degrees.
29. The system of any of examples 26-28 wherein the moveable flap is perpendicular to the base portion when in the first position and parallel to the base portion when in the second position.
30. The system of any of examples 25-29 wherein the moveable flap is composed of a flexible material.
31. Thy system of example 30 wherein the moveable flap is composed of silicone.
32. The system of any of examples 26-29 wherein the moveable flap is hingedly coupled to the base portion.
33. The system of any of examples 25-32 wherein the moveable flap is biased toward the first position.
34. The system of any of examples 25-33 wherein the actuator and the moveable flap are discrete components.
35. A method of controlling fluid flow through an implanted shunt, the method comprising:
   heating a shape memory actuator above a transition temperature,
   wherein heating the shape memory actuator above the transition temperature causes a first portion of the actuator to slide within a slot oriented within a plane parallel to a plane of the actuator, and
   wherein the slot causes the first portion to move in both—
      a horizontal direction parallel to the plane of the actuator, and
      a vertical direction perpendicular to the plane of the actuator.

36. The method of example 35 wherein heating the shape memory actuator includes (i) heating a first actuation element of the shape memory actuator to cause the first portion to move in both the horizontal direction and the vertical direction toward an aperture, (ii) heating a second actuation element of the shape memory actuator to cause the first portion to move in both the horizontal direction and the vertical direction away from the aperture, or (iii) both (i) and (ii).

37. The method of example 35 or example 36 wherein the slot causes the first portion of the actuator to simultaneously move in both the horizontal direction and the vertical direction.

38. The method of example 37 wherein the slot extends between a first end portion having a first width and a second end portion having a second width greater than the first width such that a width of the slot increases from the first end portion to the second end portion.

### Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the intraocular shunts described herein may be combined with any of the features of the other intraocular shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. An adjustable shunting system for shunting fluid from a first body region to a second body region within a patient, the system comprising:
a shunting element configured to extend at least partially between the first body region and the second body region of the patient;
a channel extending at least partially through a length of the shunting element between the first body region and the second body region, the channel including an aperture;
a flow control plate having a slot therein, the slot extending between a first end portion having a first width and a second end portion having a second width greater than the first width such that a width of the slot increases from the first end portion to the second end portion, wherein the first end portion is at least partially vertically aligned with the aperture; and
an actuator at least partially extending between the slot and the aperture, wherein the actuator includes a first portion configured to slidably engage the slot and a second portion configured to releasably engage the aperture.

2. The system of claim 1 wherein the actuator is moveable between (a) a first position in which the first portion of the actuator is adjacent the first end portion of the slot and (b) a second position in which the second portion of the actuator is adjacent the second end portion of the slot, and wherein:
in the first position, the second portion of the actuator at least partially blocks fluid flow through the aperture, and
in the second position, the second portion of the actuator does not block fluid flow through the aperture.

3. The system of claim 2 wherein the slot is configured to direct the second portion of the actuator toward the aperture as the actuator is moved from the second position toward the first position.

4. The system of claim 2 wherein the slot is configured to direct the second portion of the actuator away from the aperture as the actuator is moved from the first position toward the first position.

5. The system of claim 2 wherein the first portion and the second portion of the actuator move (i) in a horizontal direction parallel to a plane of the flow control plate and (ii) a vertical direction normal to the plane of the flow control plate.

6. The system of claim 2 wherein the actuator is selectively actuatable to move between the first position and the second position, and wherein the actuator is configured such that, following actuation, the actuator retains its position.

7. The system of claim 1 wherein the slot is an indentation in the flow control plate.

8. The system of claim 1 wherein the slot is an opening extending between opposing surfaces of the flow control plate.

9. The system of claim 1 wherein the slot is formed between one or more ridges extending from a surface of the flow control plate.

10. The system of claim 1 wherein the slot is tear-drop shaped.

11. The system of claim 1 wherein the slot includes a first side wall and a second side wall, and wherein the first side wall and the second side wall are straight.

12. The system of claim 1 wherein the slot includes a first side wall and a second side wall, and wherein the first side wall and the second side wall are curved.

13. The system of claim 1 wherein the slot includes one or more frictional elements configured to at least partially restrict movement of the first portion of the actuator within the slot.

14. The system of claim 1 wherein the actuator includes a sealing assembly, and wherein the sealing assembly includes the first portion and the second portion.

15. The system of claim 14 wherein the first portion of the sealing assembly and the second portion of the sealing assembly form a single, integral component.

16. The system of claim 14 wherein the sealing assembly has a spherical shape, and wherein the first portion is a first hemispherical portion of the spherical shape and the second portion is a second hemispherical portion of the spherical shape.

17. The system of claim 16 wherein the actuator further comprises a gating element having a cavity extending therethrough, and wherein the sealing assembly is positioned within the cavity and configured to rotate relative to the cavity.

18. The system of claim 14 wherein the first portion of the sealing assembly and the second portion of the sealing assembly are distinct, separate components.

19. The system of claim 18 wherein the first portion is composed of a first material, and wherein the second portion is composed of a second material different than the first material.

20. The system of claim 19 wherein the first material has a higher durometer than the second material.

21. The system of claim 1 wherein the actuator includes a sealing assembly and a gating element, and wherein the sealing assembly includes the first portion and the gating element includes the second portion.

22. The system of claim 21 wherein the actuator includes a recess and the sealing assembly comprises a spherical shape, and wherein the sealing assembly sits at least partially within the recess and is configured to rotate relative to the recess.

23. The system of claim 1 wherein the actuator includes a sealing assembly and a gating element, and wherein the sealing assembly is configured to rotate relative to the gating element.

24. The system of claim 1 wherein the system is configured to shunt aqueous from an anterior chamber of the patient's eye to a target outflow location within the patient.

25. An adjustable shunting system for shunting fluid from a first body region to a second body region within a patient, the system comprising:
a shunting element configured to extend at least partially between the first body region and the second body region of the patient, the shunting element having a channel extending at least partially therethrough;
an aperture configured to permit fluid to flow into or out of the channel;
a moveable flap proximate the aperture; and
an actuator configured to selectively move the moveable flap from a first position in which it does not block the aperture to a second position in which it at least partially blocks the aperture.

26. The system of claim 25 wherein the moveable flap extends from a base portion, and wherein the base portion defines the aperture.

27. The system of claim 26 wherein, when the moveable flap is in the first position, an angle between the moveable flap and the base portion is between about 60 degrees and 120 degrees.

28. The system of claim 26 wherein, when the moveable flap is in the second position, an angle between the moveable flap and the base portion is less than about 10 degrees.

29. The system of claim 26 wherein the moveable flap is perpendicular to the base portion when in the first position and parallel to the base portion when in the second position.

30. The system of claim 25 wherein the moveable flap is composed of a flexible material.

31. Thy system of claim 30 wherein the moveable flap is composed of silicone.

32. The system of claim 26 wherein the moveable flap is hingedly coupled to the base portion.

33. The system of claim 25 wherein the moveable flap is biased toward the first position.

34. The system of claim 25 wherein the actuator and the moveable flap are discrete components.

35. A method of controlling fluid flow through an implanted shunt, the method comprising:
heating a shape memory actuator above a transition temperature,
wherein heating the shape memory actuator above the transition temperature causes a first portion of the actuator to slide within a slot oriented within a plane parallel to a plane of the actuator, and wherein the slot causes the first portion to move in both—
a horizontal direction parallel to the plane of the actuator, and
a vertical direction perpendicular to the plane of the actuator.

36. The method of claim 35 wherein heating the shape memory actuator includes (i) heating a first actuation element of the shape memory actuator to cause the first portion to move in both the horizontal direction and the vertical direction toward an aperture, (ii) heating a second actuation element of the shape memory actuator to cause the first portion to move in both the horizontal direction and the vertical direction away from the aperture, or (iii) both (i) and (ii).

37. The method of claim 35 wherein the slot causes the first portion of the actuator to simultaneously move in both the horizontal direction and the vertical direction.

38. The method of claim 37 wherein the slot extends between a first end portion having a first width and a second end portion having a second width greater than the first width such that a width of the slot increases from the first end portion to the second end portion.
